(19) United States
(12) Patent Application Publication
(10) Pub. No.: US 2018/0206995 A1
(43) Pub. Date: Jul. 26, 2018

(54) ARTIFICIAL CARTILAGE CAPABLE OF SUPPLEMENTING JOINT-ELECTRICITY

(71) Applicant: Sue-May KANG, Taipei City (TW)

(72) Inventor: Sue-May KANG, Taipei City (TW)

(21) Appl. No.: 15/876,291

(22) Filed: Jan. 22, 2018

(30) Foreign Application Priority Data
Jan. 25, 2017 (TW) 106124150

(51) Int. Cl. A61F 2/30

(52) U.S. Cl. CPC A61F 2/30756; A61F 2/30942; A61F 2002/3084; A61F 2002/30087; A61F 2002/30971; A61F 2/30771

(57) ABSTRACT

An artificial cartilage was invented based on JOINT-ELECTRICITY THEORY that first in the world created in 2010, and verified in 2011, both by the present inventor, Sue-May Kang. The said artificial cartilage has sleek joint-surface and individualized appropriate shape, is made of proper piezoelectric material, and to be placed into the indication-joints, including at least the natural joints with lack or defect in cartilage and artificial joints, those suffering from impairment in generating Joint-Electricity. After surgical placement (yet, any surgery is not included in the present invention), it can be subjected to the dynamic force within the said joint in its effective range, and thus, continuously generate intra-articular electricity (is the so-called Joint-Electricity), and consequently supplements the wanted Joint-Electricity to the said joint and its related muscles. Using the said artificial cartilage can remarkably reduce the pains, increase muscular strength, and improve motor functions, when comparing to that without.

### FIELD OF THE INVENTION

The present invention is related to a medical object that can generate Joint-Electricity after it has been implanted into the indication-joint, and consequently achieves the effect of supplementing Joint-Electricity (yet, any surgery is not included in the present invention). The said Joint-Electricity is defined in the intra-articular electricity that generated by the joint-cartilage, and is required by the joint-structure tissues of the said joint and its related muscles for maintaining their normal state. More particularly, the present invention is an artificial cartilage that is made of the proper material with the property at least of piezoelectricity, which generates electricity when it is subjected to the dynamic force. Its indication-joints include artificial joints, and the natural joint those with lack or defect in cartilage, those with impairment in generating Joint-Electricity. After it has been surgically placed into the said indication-joint, it can be subjected to the dynamic force within the said joint, and generate intra-articular electricity (is the so-called Joint-Electricity) that achieves the effect of supplementing the wanted Joint-Electricity to the joint-structure tissues of the said joint and its related muscles.

### PRIOR ART

A joint in the human body, as shown in FIG. 1, is typically a junction of a plurality of bones, G1 and G2. It includes cartilage C1, and, C2, at the end of each of the bones, G1, and, G2, respectively. The joint-cartilage has a smooth joint surface and is common known to be composed essentially of collagen fibers of dense connective tissues. The cartilage C1, and, C2, is with its bottom end tightly attached to the corresponding end of bone, G1, and, G2, respectively. When compressed, both the cartilage C1 and C2 are deformed slightly to at least mitigate the pressure between the bones G1 and G2. Neither innervated, nor supplied nutrition with blood vessel, the cartilages stay in long term normal condition only when there is adequate joint exercise (those performed with proper bony alignment). However, pathology may develop in human joints, and vary greatly in cause. Particularly, those involving cartilage damage demand medical treatments and if cannot obtain optimal results, may cause agonizing pains, decreasing intra-articular spaces, restricting the range of motion, decreasing muscular strength, and developing motion difficulties. Further, the joint deformity occurs such that the forces between the bones G1 and G2 are transmitted in improper directions, resulting in a vicious circle involving further lesions including those on the said cartilage, and decreasing motor functions. If it cannot be improved through rehabilitative treatments, applicable surgical treatments can include cartilage regeneration, and cartilage replacement (with an artificial cartilage or prosthetic cartilage, or the so-called biomimetic cartilage). More severe cases may require implanting an artificial joint with a try to relatively ameliorate the existing condition and relieve pain.

However, it appears that the artificial cartilages or so-called biomimetic cartilages nowadays those implanted in the natural joint serve only as a cushion between the adjacent ends of two bones in the joint, further, the artificial joint nowadays are lack of any cartilage. Both of the artificial joint and the artificial cartilage in the prior art cannot generate intra-articular electricity, or, Joint-Electricity, after implanting them into human body. Hence, following the surgical procedure involving the usage of either of them must result suffering from severe pains (in fact, most patients need a long term and high dosage of painkillers). Further, there are difficulties in voluntary motions over the said operated joint. They suffer from further pains and difficulties during their motion therapies. In fact, most patients need extra dosage of painkillers during their motion therapy sections. Further, after their long term and painstaking motion therapies there are minimal gains in muscular strength and voluntary motion ability that slows down the recovery of voluntary motor function that they have expected. Furthermore, their artificial joints or artificial cartilages in use are prone to be soon worn-out (most of athlete cannot successfully resume their careers in sport after the said operations). High ration of the operated joints those stated above required to have repeated surgeries. Therefore, the issue to be addressed by the present invention is to provide an effective medical object to solve the problems of the prior art those stated above. This medical object is called “an artificial cartilage capable of supplementing Joint-Electricity”, and to be described at the following.

### DISCLOSURE OF THE PRESENT INVENTION

NEW THEORY THAT THE PRESENT INVENTION BASES: To solve the aforesaid problems of the prior art, the artificial cartilage of the present invention was invented based on JOINT-ELECTRICITY THEORY, which was hypothesized in 2010, and verified in 2011, both by the present inventor, Sue-May Kang. The parts of JOINT-ELECTRICITY THEORY those related to the present invention include at least the following. (1). Joint can generate intra-articular electricity (called Joint-Electricity), due to joint-cartilage is with the property of piezoelectricity, and that it is constantly subjected to the dynamic force within the said joint. Further, joint can continuously generates Joint-Electricity when the cartilage can be constantly subjected to the said dynamic force in its effective range. This is due to that joint-cartilage, being one of the piezoelectric material, has a certain range of piezoelectric reactivity, and generates Joint-Electricity only when the dynamic force within the said joint in the said range of piezoelectric reactivity, thus, this range is called its effective range. Normal joint can continuously generate Joint-Electricity, due to it is always with its intra-articular dynamic force in the said effective range. In other words, normal joint has the said range of the dynamic force within the range of the piezoelectric reactivity of its own joint-cartilage, or joint-cartilage in normal joint has a range of piezoelectric reactivity larger than the range of the dynamic force within the said joint. (2) The said Joint-Electricity can nurture the neighboring tissues of the said cartilage (called “its neighboring tissues” in the present text), which, at least include both the joint-structure tissues (e.g. joint capsule, ligaments, and tendons) of the said joint, and its related muscles (those connecting to the said tendons and those overlapping the said joint). (3). All skeletal muscles require to be nurtured by the said Joint-Electricity. First, the said joints defined in the said JOINT-ELECTRICITY THEORY include typical joints and untypical joints. The said typical joints include movable joints, slightly movable joints, and non-movable joints. The said non-movable joints included in the present theory are especially those before their cartilages becoming fibrous tissue. The said untypical joint was defined in the said theory by the present inventor, Sue-May Kang, and is stated in the following. The cartilage that located at the margin of a bone, but without the structure of typical joint, also can generate electricity when it is subjected to the dynamic force that comes from the instantly integrated result of the pull from the connecting muscles, and/or the compression from the tissues at the superficial layers and/or the objects contacting to its superficial layer. The electricity that they generated is also called Joint-Electricity. The sites of the said cartilages are called untypical joints. These joints are also included in the said JOINT-ELECTRICITY THEORY, because they are also with cartilage to generate electricity, which can nurture its neighboring tissues alike the Joint-Electricity generated from typical joints. However, they are not included in the indication-joints in the present application of the invention. Second, the said related muscles are classified with the following definitions. The muscles those with both ends each connecting to a joint that defined in the JOINT-ELECTRICITY THEORY, are nurtured by the Joint-Electricity that generated from the said two joints, and are called two-joint nurturing muscles, whereas, those only with one end connecting to a joint defined in the JOINT-ELECTRICITY THEORY, are nurtured by that from the said joint, and is called one-joint nurturing muscles. On the other hand, the muscles those with both ends located at the sites not a joint are supplemented their nurturing requirement by the Joint-Electricity that generated from the nearby joint(s), and are called neighbor-nurturing muscles. Yet, there are few neighbor-nurturing muscles in human body. Joint-Electricity, nevertheless, is the most important resource for nurturing the skeletal muscles in the human body, especially those on extremities. Thus, it is manifested that joint-cartilage can normally continuously generate Joint-Electricity is very important. (4) The Joint-Electricity that generated from the neighboring joints of the affected joint, when is in a mount large enough, can, in some degree, supplement the nurturing requirement of the neighboring tissues of the said affected joint. Therefore, the only possibility for the artificial cartilage and/or artificial joints in the prior art (they cannot generate Joint-Electricity) to have any acceptable result, requires the motion therapy that have involved the effective training of the correct usage of the nearby healthy joints for them to generate large enough Joint-Electricity to, in some degree, supplement the wanted Joint-Electricity for the requirement of the said affected joint. However, only few cases can achieve the success of this training. (5). The term of “nurture” in the phase of “Joint-Electricity can nurture the neighboring tissues of the cartilage”, or, “its neighboring tissues require to be nurtured by the said Joint-Electricity”, means that the said Joint-Electricity can be accepted by the said tissues, and if the said Joint-Electricity is sustained and enough, will have the following “nurturing effects”: keeping their lengths and tensions in normal, getting their muscles ready for the ongoing contraction that maintains the muscular strength and voluntary motion abilities in normal, remaining the said joint and its neighboring tissues in healthy, and maintaining the voluntary motor (motion) function normal at when the nervous system of the subject is still intact. Further, the joint cartilage can continuously generate Joint-Electricity is the basis for the generated Joint-Electricity can be in the enough amount. In other words, a joint can generate a large amount of Joint-Electricity requires the adequate motion training on the basis of that the said joint can continuously generate Joint-Electricity. On the contrary, when the Joint-Electricity that generated from the said cartilage (in the said joint) is not continuous for a period due to whatever reason (so that the said Joint-Electricity is manifested “not enough”), the said tissues would be with the contrary condition to those stated above, and require the supplementation of the said electricity. (6) The said dynamic force within the said joint that cooperating to the said joint-cartilage(s) for generating Joint-Electricity is the instant sum of those come from at least two resources. All typical joints, being with the feature at least of their cartilages located between multiple bones, and have joint capsules or ligaments outside, have made the coming forces integrated to be intra-articular reaction force, which, then, becomes the major source of the said dynamic force for the typical joint. Further, there is another resource of the said dynamic force, it is coming from the instant sum of blood pulses, breathing rhythm, and other motions those coming from viscera (such as gut). This kind of dynamic force is called vital-organ mechanism, and is especially dominant at when the subjects are sleeping. The most important, at any time, the instant dynamic force for the typical joints is the instant sum of that coming from both the said vital-organ mechanism and intra-articular reaction force. Therefore, for simply stating in the following text of the present invention, the term “dynamic force” is used to call the instant sum of forces those stated above that responsible for cooperating with joint-cartilage in generating Joint-Electricity. (7) According to JOINT-ELECTRICITY THEORY, the alignment of the joint related structures should be correct, in order for every part of the joint-cartilage can be subjected to the dynamic force within the said joint at least in correct direction during the motions in every direction, and is the most important basis for ensuring the said dynamic force within the said joint in its normal range, which can be definitely stated, in its effective range. Based on the basis that stated above, that contributes to the said artificial cartilage of the present invention can be subjected to the dynamic force in its effective range is that the artificial cartilage has been made in the appropriate piezoelectric material that has large enough range of piezoelectric reactivity, which algorithm has been described in the first point of the present section ([0004]-(1)), and its technical means is described in the next section “SUMMARY OF THE TECHNICAL MEANS OF THE PRESENT INVENTION”, [0005], and other paragraphs ([0007] and [0022]). Wherein, the said correct alignment can be achieved through not only the correct alignment of the joint-structure excepting cartilage (bones, capsule, ligaments, and joint space) that achieved during the proper surgery placing the said artificial cartilage to the said joint, but also the individualized appropriate shape of the said artificial cartilage (that contributing to a correct and complete shape of the healed cartilage) that ensuring none-disturbing the said correct alignment. So that implanting an artificial cartilage to manage the problems related to the defect or lack in cartilage, should use the artificial cartilage that not only made of the proper piezoelectric material, but also with individualized appropriate shape. (8) The said proper surgery is defined in the surgery that has recovered the normal joint structure completely and correctively, which includes regaining the correct alignment between the adjacent bones of the said joint (if required, cutting or filling the bone end), recovering the structure of the said joint-structure tissues (capsule, ligament, and tendons) to be complete and correct (reserving all the related tissues, if required, repairing them fully), normalizing the joint space related to every part of the said healed cartilage (keeping the lengths of every part of the capsule and ligaments as normal as when they are normal, if required, repairing or reconstructing them), and making the joint fluid being with adequate amount and quality (if required, supplementing with appropriate joint-fluid in adequate amount (including artificial joint-fluid)). Thus, the said proper surgery is the basic condition for the dynamic force within every part of any of the indication-joint can be correct in both direction and its normal range (alike that when the joint is in normal condition, or that estimated from the contralateral side), yet, any surgery is excluded from the present invention. It should be mentioned that the said proper surgery is thought have been performed similarly during the processes of implanting any kind of artificial cartilage, and during the comparisons within the effects of the usages of different kinds of artificial cartilage in the text for the present invention. Therefore, the said surgical methods will not be repeated described in the following sections, except some necessary part of it in some necessary section(s).

SUMMARY OF THE TECHNICAL MEANS OF THE PRESENT INVENTION: For solving the problems in the prior art, the technical means that provided by the present invention is a special medical object, called “artificial cartilage capable of supplementing Joint-Electricity”, which was developed based on JOINT-ELECTRICITY THEORY that created by the present inventor, Sue-May Kang. The definition of the said Joint-Electricity is briefly described in the following: Joint-Electricity is the intra-articular electricity that generated by the joint-cartilage as which has the property of piezoelectricity and can be constantly subjected to the normal dynamic force within the said joint, further, it is required by the joint-structure tissues of the said joint and its related muscles for maintaining their normal state. The indication-Joints for using the present invention include at least the natural joints those with lack or defect in cartilage those requiring repairing or replacing its cartilage with an artificial cartilage, and, artificial joints (which, have been developed for decades, are lack of artificial cartilage), those suffering from impairment in generating Joint-Electricity. An artificial cartilage invented by the present inventor is with a sleek joint-surface, an individualized appropriate shape, made of a piezoelectric material (defined in the proper material with the property of piezoelectricity), especially a proper piezoelectric material that with the property of proper piezoelectric reactivity (its definition is described latter), and to be placed into the said indication-joint. The said “to be placed” into the said indication-joints is by the methods of surgically implanting to either the said natural joint or artificial joint, and, non-surgically placing to the artificial joint in previous of the said implanting, and then together with the said artificial joint being implanted into the target subject. Yet, the methods for the artificial cartilage of the present invention to be placed and secured into the said joint during surgery are part of the said surgery, being excluded from the present invention. Further, any surgery is excluded from the present invention. The said proper piezoelectric reactivity is defined in the said piezoelectricity is with a range of piezoelectric reactivity that is large enough that ensures that the artificial cartilage that it makes has a range of piezoelectric reactivity larger than the range of the dynamic force that expected or estimated within the target indication-joint after the proper surgery for implanting it. (the definition of the said proper surgery has been described in [0004]-(8), but, is excluded from the present invention). Thus, the artificial cartilage of the present invention is expected to be subjected to the dynamic force within the said joint in its effective range, and consequently generate Joint-Electricity continuously after it has been implanted into the said joint. Therefore, the said artificial cartilage can achieve the effect of supplementing the wanted Joint-Electricity to the joint-structure tissues of the said joint (e.g. capsule, ligaments, tendons) and its related muscles (those connecting to or overlapping the said joint). Comparing to those without using the artificial cartilage of the present invention, usage of the artificial cartilage of the present invention can remarkably reduce the pains, increase the muscle strength and voluntary motor ability, improve the motor functions, and longer sustain the health of the said artificial cartilage (and artificial joint) and its neighboring tissues. Further, adding the artificial cartilage of the present invention to the artificial joints (during the implanting surgery, or, placing it into the said artificial joint in previous of the said implanting, and afterwards implanting them together) can ensure the said artificial joint after implantation by the proper surgery can continuously generate Joint-Electricity that supplements the wanted Joint-Electricity to the joint-structure tissues of the said artificial joint and its related muscles, making the said artificial joint have greater advantage over those without adding the said artificial cartilage, and longer sustain the health of the said artificial joint. However, the methods for placing and securing the said artificial cartilage into the said artificial joint during surgery are part of the surgery, and are excluded from the present invention, whereas, the non-surgical method for placing and securing the said artificial cartilage to the artificial joint in previous of the said surgery is stated in [0013] and [0014]. Further, any surgery is not included in the present invention. Therefore, the artificial cartilage of the present invention can effectively solve the problems those stated in the PRIOR ART. The characteristic, feature, and objective of every point of the technical means of the artificial cartilage of the present invention are stated in the following paragraphs, with each paragraph for each point.

One characteristic and objective of the artificial cartilage of the present invention is to provide an artificial cartilage capable of generating Joint-Electricity, and thus, supplementing the wanted Joint-Electricity to the joint-structure tissues of the said joint and its related muscles. The said artificial cartilage is made of the piezoelectric material, to be placed into the indication-joints (artificial joints, and, the nature joints those with defect or lack in cartilage) those suffered from impairment in generating Joint-Electricity. The said placing of the artificial cartilage of the present invention into the said indication-joints include surgically implanting to the said natural and artificial joint, in addition to non-surgical placing to the said artificial joint in previous of the said implanting and then together with the said artificial joint being implanted into the target subject, yet, any surgery is not included in the present invention. Wherein, the said piezoelectric material is defined in that the material that has the property of piezoelectricity, which means that when it is subjected to the dynamic force within the said joint, it can be deformed and then generate intra-articular electricity (is the so-called Joint-Electricity). Thereby, with the said property of piezoelectricity, the artificial cartilage of the present invention can generate Joint-Electricity alike the natural joint-cartilage does (after the proper surgery implanting it into the said indication-joint, yet, the said proper surgery, has been described in [0004]-(8), is not described here, and is not included in the present invention). The generated Joint-Electricity can increase the nurturing on the joint-structure tissues of the said joint and its related muscles that achieves the effects of supplementing the wanted Joint-Electricity to the said tissues, and consequent increasing the muscular strength and voluntary motion ability comparing to that without using the present invention. Besides, the said material of the said artificial cartilage of the present invention has also the property of high biocompatibility, which means that the said material will not be rejected by, or, harm, the human body it is placed into. (however, the placing surgery is excluded from the present invention).

Another characteristic and objective of the artificial cartilage of the present invention is that the said artificial cartilage of the present invention can also be made of a proper piezoelectric material. The said proper piezoelectric material is defined in that it has the property of large enough range of piezoelectric reactivity that makes the said artificial cartilage being with large enough range of piezoelectric reactivity, which is at least larger than the range of the dynamic force that estimated in the individualized target indication-joint after the proper surgery that implanting it, yet, any surgery is not included in the present invention. Thereby, the said property of large enough range of piezoelectric reactivity of the said artificial cartilage of the present invention contributes to ensuring the said artificial cartilage of the present invention can be subjected to the dynamic force within the said joint in its effective range, and consequently continuously generate adequate amount of Joint-Electricity alike the healthy natural cartilage does that accomplishing the effect of supplementing the wanted Joint-Electricity to the joint-structure tissues of the said joint and its related muscles. Comparing to those without using the artificial cartilage of the present invention, the usage of the said artificial cartilage of the present invention ensures to achieve the effects of remarkably reducing the pains, increasing the muscular strength and voluntary motion ability of the said joint that improving voluntary motor function of the indication-subject, and longer sustaining the use life of the using artificial cartilage, and the joint using it (including the artificial joint). Besides, the said proper piezoelectric material of the said artificial cartilage of the present invention is also highly biocompatible, meaning that the said material will not be rejected by, or, harm, the human body it is placed into, (however, the placing surgery is not included in the present invention).

Yet another feature and objective of the artificial cartilage of the present invention is that the shape of the present invention is designed individually and appropriate. The designing methods include at least that according to the shape of the corresponding original healthy cartilage in the target indication-joint, when which cartilage is that to be totally replaced, or, according to at least the target joint-surface of the artificial joint of the indication-subject, when which is where the said artificial cartilage to be placed. This is for the artificial cartilage of the present invention can be with a correct and complete shape in order to be placed into and fit to the said indication-joint, and consequent preventing from the disturbance in that it can be, in its every part, subjected to the dynamic force within the said indication-joint in its effective range that obtained from both the said proper surgery (which, yet, is not included in the present invention) and special property of the artificial cartilage of the present invention, and thus, continuously generates Joint-Electricity during daily living.

Still another feature and objective of the artificial cartilage of the present invention is that it is provided with its shape ensuring individualized and appropriate that ensures never disturb the correct joint structure that contributed by proper surgery, which, yet, is not included in the present invention. This is achieved through the methods including that the said shape of the artificial cartilage of the present invention can be individually designed according to the individualized condition of defect or lack in the said cartilage. The examples include that the said artificial cartilage of the present invention is designed according to the shape complementary to the shape of the residual cartilage in the target indication-joint, in order for it can be placed and secured to the said residual cartilage to become a healed cartilage that in the shape complete and correct. However, the methods of placing and securing the present invention to the said residual cartilage and cartilage-attaching corresponding bony site are included in the said proper surgery, and are excluded from the scope of the present invention. The said complete and correct shape of the healed cartilage can avoid from disturbing that it can be, in its every part, subjected to the dynamic force within the said indication-joint in its effective range that obtained from both the said proper surgery, (yet, which is not included in the present invention) and the special property of the artificial cartilage of the present invention, and consequently naturally continuously generate Joint-Electricity during the activities in daily living.

Another feature and objective of the artificial cartilage of the present invention is, at when necessary, the said artificial cartilage can also be made of the proper material of supporting fibers, in addition to the said piezoelectric material. Wherein, the making method can be, first, making the said piezoelectric material of the artificial cartilage of the present invention into the said supporting fibers, which, then, with enough amount, are made into the said artificial cartilage of the present invention.

Another feature and objective of the artificial cartilage of the present invention is to provide the artificial cartilage of the present invention with the joint-surface in sleek. For the embodiments of the present invention those excluding the artificial cartilage of the present invention those stated in [0010] those have included supporting fibers those made of piezoelectric material of the artificial cartilage of the present invention, apart common smoothing techniques, the sleek joint-surface can be achieved through applying the nanostructurizing process to some of the material that same to that of the said artificial cartilage of the present invention, and then that after nanostructurizing is coated onto the joint-surface of the said artificial cartilage, or, it can also be achieved through applying the said nanostructurizing process to enough amount of the material of the said artificial cartilage of the present invention, and then that after nanostructurizing is made into the said artificial cartilage.

Another feature and objective of the present invention is related to the method for the embodiments of the artificial cartilage of the present invention those stated in [0010] (those have included supporting fibers those made of the piezoelectric material of the said artificial cartilage of the present invention) to have its joint-surface sleek. The sleek joint-surface can be achieved through at least, first, making the said supporting fibers in the diameter that as small as possible (the smallest diameter that the related industry instantly can make), which, then, with enough amount, are made into the said artificial cartilage of the present invention.

Still another feature and objective of the artificial cartilage of the present invention is that the artificial cartilage of the present invention can be placed into an artificial joint (either during the implanting surgery of the said artificial joint, or, in previous of the said implanting surgery and afterwards, together with the said artificial joint, being implanted into the target indication-subject) in order to make the said artificial joint become the joint capable of generating Joint-Electricity in daily living alike a natural healthy joint does, and consequently achieve the effect of supplementing the wanted Joint-Electricity to the joint-structure tissues of the said artificial joint and its related muscles. The method for placing and securing the artificial cartilage of the present invention to the artificial joint during the implanting surgery of the artificial joint is part of the method of the said surgery, and all surgeries are excluded in the present invention, whereas, the methods of placing and securing the artificial cartilage of the present invention to the artificial joint in previous of the said implanting surgery are that must be described in the present invention. The said placing and securing methods include at least, using the appropriate adhesive bonding material (the type that has included the nanostructured material is one of the examples) that suitable for both the bottom of the said artificial cartilage of the present invention and site corresponding to the said cartilage inside the said artificial joint to directly bond the said artificial cartilage to the said site inside the said artificial joint. Further, for the embodiments of the artificial cartilage of the present invention excluding those stated in [0010] (those have included supporting fibers those made of the piezoelectric material of the said artificial cartilage of the present invention) can also use the following methods: coating the material of the said artificial cartilage of the present invention to the said site inside the said artificial joint in the shape of the said artificial cartilage, and other methods. After the said artificial joint has been implanted by a proper surgery, it can generate adequate Joint-Electricity alike natural healthy joint does due to it has equipped the said artificial cartilage of the present invention, and which can be subjected to the adequate dynamic force within the said artificial joint. Yet, the method that implanting the said artificial joint to the target subject is a surgical method, and is not included in the present invention.

Still another feature and objective of the artificial cartilage of the present invention is that the embodiments of the artificial cartilage of the present invention those stated in [0010] (those have included supporting fibers those made of the piezoelectric material of the said artificial cartilage of the present invention) can also be placed into an artificial joint (either during the implanting surgery of the artificial joint, or, in previous of the said implanting surgery and afterwards, together with the said artificial joint, being implanted into the target indication-subject) in order to make the said artificial joint become the joint capable of continuously generating Joint-Electricity in daily living alike a healthy natural joint does, and consequently achieve the effect of supplementing the wanted Joint-Electricity to the joint-structure tissues of the said artificial joint and its related muscles. The method of placing and securing the said artificial cartilage to the said artificial joint during the implanting surgery of the artificial joint is part of the said surgery, and all surgeries are excluded from the present invention, whereas, the methods of placing and securing the artificial cartilage of the present invention to the artificial joint in previous of the said implanting surgery must be described in the present invention. The said methods include: using the appropriate adhesive bonding material (the type that has included the nanostructured material is one of the examples) that suitable for both the bottom of the said artificial cartilage of the present invention and site corresponding to the said cartilage inside the said artificial joint to directly bond the said artificial cartilage to the said site inside the said artificial joint, and the other methods excluding coating. After the said artificial joint has been implanted by a proper surgery, it can generate adequate Joint-Electricity alike natural healthy joint does due to it has equipped the said artificial cartilage of the present invention, and which can be subjected to the adequate dynamic force within the said artificial joint. Yet, the method that implanting the said artificial joint to the target subject is a surgical method, and is not included in the present invention.

THE IMPROVEMENTS of the artificial cartilage of the present invention over those of prior art: First, the indication-joints of the present invention further include the artificial joints (artificial joints develop till nowadays still lack cartilage), in addition to those of the prior art those mainly the natural joints those with defect or lack in cartilage, and hence, require replacing or repairing their cartilages with artificial cartilages. Both of them, based on JOINT-ELECTRICITY THEORY that the present inventor, Sue-May Kang, created, are similar in being suffered from the impairment in normally generating Joint-Electricity. The said Joint-Electricity is defined in the intra-articular electricity that generated by the joint-cartilage as which has the property of piezoelectricity and is constantly subjected to the normal dynamic force within the said joint, while the said Joint-Electricity is required by the joint-structure tissues of the said joint and its related muscles for maintaining their normal state. Second, the artificial cartilage of the present invention, being created based on the said JOINT-ELECTRICITY THEORY, has the advantage in it is made of proper piezoelectric material, after it has been implanted into the indication-joint by a proper surgery (yet, any surgery is excluded from the present invention), it ensures can be subjected to the dynamic force within the said joint in its effective range, thus, can continuously generate Joint-Electricity. The Joint-Electricity that generated by the said artificial cartilage of the present invention can, alike that generated by the natural healthy cartilage, full time and increasedly nurture the joint-structure tissues of the said indication-joint and its related muscles that accomplishes the goal of supplementing the wanted Joint-Electricity to the said tissues. This is the reason why that the said artificial cartilage of the present invention is more effective and advanced than those in prior art. Third, the present invention can also be placed and secured into the artificial joint not only during implanting surgeries, but also in previous with non-surgical methods and then implanted together with the said artificial joint (yet, the related surgeries are not included in the present invention), and hence, makes the artificial joint equipped with a cartilage that alike a natural healthy cartilage. Equipping with the artificial cartilage of the present invention that alike the healthy natural cartilage, the said artificial joint can be more alike a natural healthy joint, continuously generating Joint-Electricity, which can be alike that generated from the natural healthy joint, full time and increasedly nurture the joint-structure tissues of the said artificial joint and its related muscles, so is more effective and advanced than any artificial joint has ever been at before. Fourth, the usage of the artificial cartilage of the present invention in the indication-artificial joints, or, natural joints, has the following benefits over those without, or, those using the artificial cartilage or so-called biomimetic cartilage in the prior art. They, at least, include remarkably decreasing pains, increasing muscular strength, and easing voluntary motions, thus, remarkably more improving the motor functions in daily living, and longer keeping the health and use life of the said artificial cartilage (and artificial joint). All benefits of the artificial cartilage of the present invention those stated above can be simply stated in that usage of it has the effect of supplementing Joint-Electricity that wanted at before using it.

### BRIEF DESCRIPTION OF THE DRAWINGS

FIG. 1 This diagram schematically shows the side view of an example of human natural joints that when its cartilage C1, and, C2, healthily staying at the end of the bone G1, and, G2, respectively, inside the said joint.

FIG. 2 This diagram schematically shows the side view of one of the embodiments of the artificial cartilage of the present invention that to be placed in the indication-joint that shown in FIG. 1 when C1 in the FIG. 1 requires to be totally replaced. Wherein, 1 denotes the said artificial cartilage.

FIG. 3 This diagram schematically shows the side view of another embodiment of the artificial cartilage of the present invention that to be placed in the indication-joint that shown in FIG. 1 when C1 in the FIG. 1 requires to be repaired by the said artificial cartilage. Wherein, 1A denotes the said artificial cartilage.

FIG. 4 This diagram schematically shows the side view of another embodiment of the artificial cartilage of the present invention (that has included supporting fibers those made of piezoelectric material of the said artificial cartilage of the present invention) that can be the option of that shown in FIG. 2. Wherein, 21 schematically denotes the said supporting fibers.

### DETAILED DESCRIPTION OF THE INVENTION

BRIEF DESCRIPTION OF THE MOST BASIC RELATIONSHIP between JOINT-ELECTRICITY THEORY and the technical means of the artificial cartilage of the present invention. The present invention is an artificial cartilage capable of generating Joint-Electricity, and consequent getting the effect of supplementing the wanted Joint-Electricity. The artificial cartilage of the present invention is invented based on JOINT-ELECTRICITY THEORY that created in 2010, and verified in 2011, both by the present inventor, Sue-May Kang. The most basic relationship between the artificial cartilage of the present invention and the said theory includes at least the following. (1). Natural joint can generate intra-articular electricity (is the called Joint-Electricity), and this is due to joint-cartilage has the property of piezoelectricity, and can be constantly subjected to the dynamic force within the said joint. Further, joint can continuously generate Joint-Electricity, when the said cartilage can be constantly subjected to the dynamic force within the said joint in its effective range. So that, the artificial cartilage of the present invention is specially created to be made of piezoelectric material, especially of a proper piezoelectric material that has large enough range of piezoelectric reactivity in order to ensure the said artificial cartilage, after being implanted into the target indication-joint by a proper surgery (yet, any surgery is excluded from the present invention), can be subjected to the dynamic force within the said joint in its effective range, and consequently, alike a natural healthy joint-cartilage does, continuously generates Joint-Electricity that achieves the effect of supplementing the wanted Joint-Electricity to the joint-structure tissues of the said joint and its related muscles. Thereby, the artificial cartilage of the present invention has the indication-joints including artificial joints (they develop till now, are still lack of artificial cartilage) and the natural joints those with lack or defect in cartilage, those have impairment in continuously generating Joint-Electricity. (2). Base on the said JOINT-ELECTRICITY THEORY, the Joint-Electricity that generated by normal natural cartilage has the function of nurturing its neighboring tissues, including at least the joint-structure tissues (e.g. joint-cartilage, ligament, and tendons) and its related muscles (those connecting to the said tendons and those overlapping the said joint). The said nurturing means that the said Joint-Electricity has been accepted by the said tissues, and if the amount of the said Joint-Electricity is enough, the length and tension of the said tissues are maintained, the muscles are gotten ready for the contraction that to perform, so that the muscle strength and voluntary motion ability are kept normal, under the nervous system is intact. On the contrary, when the Joint-Electricity generated from the natural cartilage is not enough due to any reason for a long enough period, the said nurturing effect would be hindered, and consequent the shortening or tension-altering of the said tissues, pains, decreasing muscular strength and voluntary motion abilities, and impaired functional motor functions. So that, when the lengths of the said tissues have been shorten before the surgery and without correction during the said surgery, they cannot be recovered its length, or, normalized immediately with the usage of the present invention in the said surgery. This is due to that the said artificial cartilage still cannot in its every part be subjected to the dynamic force within the said joint in its effective range, which hinders the said artificial cartilage to continuously generate an adequate amount of Joint-Electricity, and consequent the hindering on the said nurturing effect. Therefore, the proper surgery that has been briefly described its content in [0004]-(8) is required, not only for recovering the normal structure (or normal condition) of the said joint, but also for the said artificial cartilage to immediately continuously generate Joint-Electricity, which, in turn, is required for immediately and long term keeping the afterwards-health of the said joint (keeping the normal joint-structure after the said implanting). The said proper surgery is similarly important for all the operations related to joint. It is thought that any kind of artificial cartilage has been implanted by the said proper surgery. This, thus, is the basis of comparing the effects of the usage of the different types of artificial cartilage in the text of the present application. So that, the content of the said proper surgery is not repeatedly described in the following text, further, any surgery is excluded from the present invention. Thereby, the individualized and correct shape of the artificial cartilage is also required for ensuring it does not hinder the correct joint structure (including correct alignment) that obtained by the said proper surgery. This is for ensuring every part of it can be subjected to the dynamic force within the said joint in its effective range in order to contributing to continuously generate Joint-Electricity for supplementing the wanted Joint-Electricity to the joint-structure tissues of the said joint and its related muscles. The main characteristics of the artificial cartilage of the present invention those stated above contributes to the advanced effects of the usage of the artificial cartilage of the present invention, comparing to that without, include at least remarkably reducing pains, increasing muscular strength and voluntary motion abilities, promoting motor functions, and keeping the health of the said artificial cartilage and the said joint (including the artificial joint). The following paragraphs show some of the embodiments of the artificial cartilage of the present invention.

The present invention, based on the JOINT-ELECTRICITY THEORY that created by the present inventor, Sue-May Kang, provides an artificial cartilage capable of generating Joint-Electricity, and thus, getting the effect of supplementing Joint-Electricity to its neighboring tissues. In its embodiments, it has sleek joint-surface, an individualized appropriate shape, and to be placed into the indication-joint (the natural human joint that with defect or lack in cartilage, or, artificial joint) so as to be subjected to the dynamic force within the said joint, and continuously deformed by the said dynamic force that generating intra-articular electricity (is the so-called Joint-Electricity). The said “be placed into the said indication-joints” is by the methods including surgically implanting to either the said natural joint, or the said artificial joint, and non-surgically placing to the said artificial joint in previous of the implanting surgery of the artificial joint, and afterwards are surgically implanted together with the said artificial joint. Yet, any surgery is excluded from the scope of the present invention. In an embodiment of the present invention, the artificial cartilage of the present invention is made of the piezoelectric material that defined in the proper material that with the property of piezoelectricity. Wherein, the said piezoelectric material of the artificial cartilage can also demonstrate piezoelectricity effect within the indication-joint after placing surgery (yet, the all surgeries are excluded from the present invention). Please note that the term piezoelectricity effect as used herein refers to “direct piezoelectricity effect”, meaning that when it is deformed by the dynamic force, the mechanical energy is converted into electric energy. Therefore, after the said artificial cartilage of the present invention has been placed into the target indication-joint by a proper surgery (which has been described in [0004]-(8), and is excluded from the present invention), the said artificial cartilage can be deformed at subjecting to the normal dynamic force within the said joint, and generates intra-articular electricity (is the so-called Joint-Electricity) through the said piezoelectricity effect of the said artificial cartilage. Thereby, it achieves the effect of supplementing the wanted Joint-Electricity to the neighboring tissues of the said artificial cartilage that increases the nurturing of the said tissues. Comparing to that without using the artificial cartilage of the present invention, usage of the said artificial cartilage of the present invention can increase the muscular strength, decrease the pains, and improve the voluntary motor ability. Besides, the said piezoelectric material of the artificial cartilage of the present invention is also highly biocompatible, meaning that the said material will not be rejected by, or, harm, the human body it is placed into, (however, the placing surgery is excluded from the present invention).

The artificial cartilage of the present invention, in another embodiment, if necessary, is characterized in made of a proper piezoelectric material. The said proper piezoelectric material is defined in that the material that has a proper range of piezoelectric reactivity that can make the said artificial cartilage have a proper range of piezoelectric reactivity. The said proper range of piezoelectric reactivity of the said artificial cartilage of the present invention means that it is not only with the direct piezoelectricity property, but also has a large enough range of piezoelectric reactivity, which at least larger than the estimated range of the dynamic force within the said indication-joint of the indication-subject (after the proper surgery that implanting it into the said indication-joint, yet the said surgery is not included in the present invention). The said property of large enough range of piezoelectric reactivity of the said artificial cartilage of the present invention ensures the said artificial cartilage can be subjected to the dynamic force within the said indication-joint in its effective range, and consequently, can, alike the natural normal cartilage in a normal joint does, continuously generate Joint-Electricity (after placing it into the said indication-joint by the proper surgery, which is not included in the present invention). The generated Joint-Electricity, thus, is alike that generated by the normal natural cartilage, can full time and increasedly nurture the joint-structure-tissues of the said joint and its related muscles, obtaining the goal of supplementing the wanted Joint-Electricity to the said tissues. Thus, using the said artificial cartilage of the present invention, comparing to those without using the present invention, can remarkably reduce the pains, increase the muscular strength and voluntary motion ability, and remarkably improve the voluntary motor functions and longer sustain the use life of both the said artificial cartilage of the present invention and the joint using it (including artificial joint). Besides, the said proper piezoelectric material of the artificial cartilage of the present invention is also highly biocompatible, meaning that the said material will not be rejected by, or, harm, the human body it is placed into. (however, the placing surgery is excluded from the present invention).

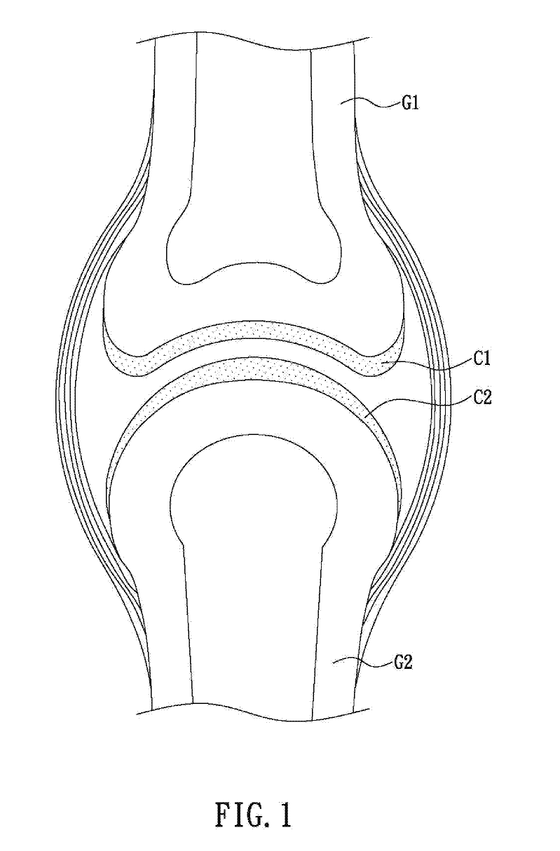

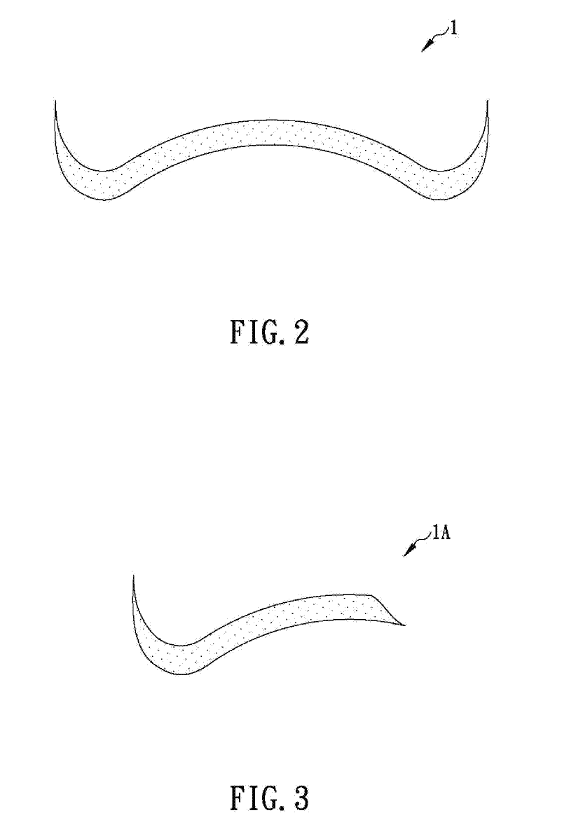

Further, the shape of the artificial cartilage of the present invention can be individualized designed to be appropriate according to the clinical requirement of the individual target indication-joint. For example, in one of the embodiments of the present invention, (see FIG. 1, which denotes an example of a natural joint with cartilage C1 and C2 inside, and FIG. 2, which denotes the artificial cartilage of the present invention designed for replacing the cartilage C1 in the said joint in FIG. 1), when the cartilage C1 in the FIG. 1 is the cartilage in the indication-joint and is that to be totally replaced, the shape of the artificial cartilage of the present invention (1 in the FIG. 2) that designed for replacing it can be designed with methods including imitating the shape of the original healthy cartilage as that shown in C1 in FIG. 1. That is, the shape of the said artificial cartilage of the present invention in FIG. 2 can be same to that of C1 in FIG. 1. Similarly, in the embodiment that the said artificial cartilage is to be placed into an artificial joint, it's shape is designed individualized and appropriate with the methods at least include according to the shape of the joint-surface of the said artificial joint. But, if necessary, as in another embodiment of the artificial cartilage of the present invention, when the cartilage C1 has been only partially damaged and requires to be repaired by the artificial cartilage of the present invention, which shape could be individually designed to be appropriate according to the problem related to the defect or lack in the cartilage of the indication-joint, the designing methods include according to the complementary shape of the residual cartilage in the target indication-joint that forms the said artificial cartilage in the shape as shown in 1A in FIG. 3. The said artificial cartilage 1A (in FIG. 3) in this embodiment of the present invention can be placed and secured to the residual part of the damaged cartilage and corresponding bony part of the articular-surface of the bone end in the said indication-joint to become the healed-cartilage that with complete and correct shape that same to the shape of its original healthy cartilage as shown in C1 in FIG. 1. Yet, the said placing and securing process is part of the surgical processes, which are all excluded from the present invention. Nevertheless, the complete and correct shape of the said healed-cartilage (that contributed by the present invention including that in full shape, or, that in partial and complementary shape, or, . . . ) contributes to ensuring that it does not disturb the optimal joint space or correct alignment of the joint structure that contributed by the said proper surgery (yet, which is not included in the present invention). Thus, the said individualized appropriate shape of the said artificial cartilage allows every part of the said artificial cartilage during the motions in every direction can be subjected to the dynamic force within the said joint in correct direction (that contributed by the said proper surgery), in addition to, effective range in amount (that contributed by both the said proper surgery and the special property of the material that the artificial cartilage of the present invention made of)(see [0004], for its reasoning). Thereby, the said artificial cartilage can continuously generate Joint-Electricity to achieve the effect of supplementing the wanted Joint-Electricity to the joint-structure tissues of the said indication-joint and its related muscles. Yet, any surgery is excluded from the present invention.

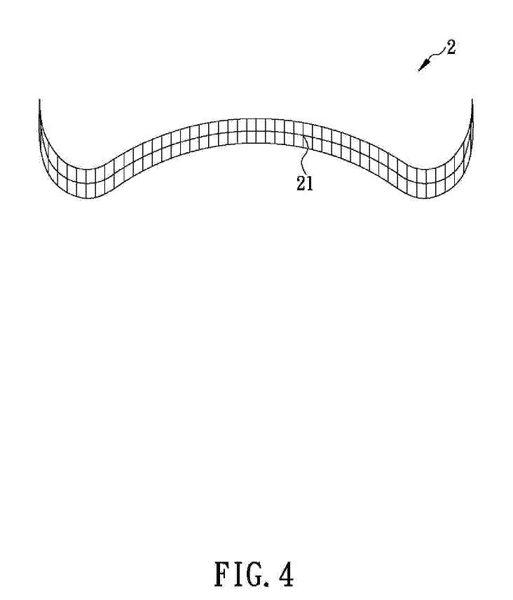

Further, the artificial cartilage of the present invention, in another embodiment, if necessary, can be made of the proper material of supporting fibers, in addition to the said piezoelectric material. Wherein, the said supporting fibers have the property of proper flexibility that allows them to be deformed with the deforming of the said artificial cartilage at reacting to the dynamic force within the indication-joint. The making method can be, first, making the said piezoelectric material of the said artificial cartilage of the present invention into the said fibers, which, then, with enough amount, are made into the said artificial cartilage of the present invention in the shape according to the individualized shape of the artificial cartilage of the target indication-joint of the target subject (as shown in 1 in FIG. 2), to make the said artificial cartilage in the shape and size as that expected to be, and become that illustrated schematically in 2 of FIG. 4, yet, the arrangement or organization of the said supporting fibers is not limited to that shown as 21 in the FIG. 4).

The artificial cartilage of the present invention is provided with its joint-surface in sleek, wherein, for the embodiments of the artificial cartilage of the present invention those excluding those stated in [0024] (those have included supporting fibers those made of the piezoelectric material of the said artificial cartilage of the present invention), the methods those used for making the joint-surface sleek include the following. For example, using the common surface smoothing techniques, or, applying a nanostructurizing process to some of the material same to that of the said artificial cartilage, then that after nanostructurizing is coated onto the said joint-surface of the said artificial cartilage of the present invention, or, applying the nanostructurizing process to enough amount of the material of the said artificial cartilage of the embodiments of the present invention, then that after nanostructurizing is made into the said artificial cartilage. This is for the purpose of having the artificial cartilage of the said embodiments of the present invention to have a sleek joint-surface same to the natural healthy cartilage for minimizing the possible mechanical friction, and consequently is not prone to be worn-out or broken, in order to be long term used after surgically placing it into the indication-joint. However, any surgery is not included in the present invention.

The artificial cartilage of the present invention is provided with its joint-surface in sleek, wherein, for the embodiments those stated in [0024] those have included supporting fibers those made of the piezoelectric material of the said artificial cartilage of the present invention, the methods those used for making the joint-surface sleek include at least, first, manufacturing the said fibers with the said material of the artificial cartilage of the present invention with its diameter as small as possible (as small as the instant industrial can make), and then, making the said small-diameter fibers to form the said artificial cartilage of the present invention in the shape appropriate to the target artificial cartilage of the present invention for the target indication-joint. This is for the purpose of having the said embodiments of the artificial cartilage of the present invention to be with sleek joint-surface same to the natural healthy cartilage for minimizing the possible mechanical friction, and consequently is not prone to be worn-out or broken, in order to be long term used after placing it into the indication-joint. However, the surgery for placing the present invention into the said indication-natural-joint, and/or indication-subject is not included in the scope of the present invention).

The so-called human joints those included in the said indication-joints include the joints those of movable (or, called diarthrosis), slightly movable (or, called amphiarthrosis), and non-movable (or, called synarthrosis). The said non-movable joints in the said indication-joints are those for growing needs, such as fonticulus anterior and fonticulus posterior of a child.

The embodiments of the artificial cartilage of the present invention include that the said artificial cartilage of the present invention can be placed into an artificial joint (either during the implanting surgery of the said artificial joint, or, in previous of the said implanting surgery and afterwards, together with the said artificial joint, being implanted into the target indication-subject) in order to make the said artificial joint become the joint that can generate adequate Joint-Electricity in daily living alike a healthy natural joint does, and consequently achieve the effect of supplementing the wanted Joint-Electricity to the said joint and its related muscles. In one embodiment, the said placing and securing of the said artificial cartilage to the said artificial joint is during the implanting surgery for implanting the said artificial joint into the target subject. However, the method of placing and securing the said artificial cartilage into the said artificial joint during the implanting surgery for implanting the said artificial joint is part of the said surgery, and is not included in the present invention. Further, in another embodiment, the said artificial cartilage of the present invention also can be placed into and secured to the artificial joint before the said implanting surgery of the said artificial joint, and afterwards, it, together with the said artificial joint, can be implanted into the target indication-subject. The methods of placing and securing the said artificial cartilage of the present invention to the artificial joint before the implanting surgery of the said artificial joint include adhesive bonding, coating (only suitable for the embodiments of the present invention those excluding those stated in [0024] those have included supporting fibers those made of the piezoelectric material of the said artificial cartilage of the present invention), and other methods. For example, using the appropriate adhesive bonding material (the type that has included the nanostructured material is one of the examples) that suitable for both the bottom of the said artificial cartilage of the present invention and joint-surface of the said artificial joint to directly bond the said artificial cartilage to the joint-surface of the said artificial joint. For the embodiments of the present invention excluding those stated in [0024] those have included supporting fibers those made of piezoelectric material of the said artificial cartilage of the present invention can also use the following methods: coating the material of the said artificial cartilage of the present invention to the said joint-surface of the said artificial joint in the shape of the said artificial cartilage, and other methods. After the said artificial joint has been implanted by a proper surgery, it can continuously generate Joint-Electricity alike natural healthy joint does due to it has equipped the said artificial cartilage of the present invention, and which can be subjected to the dynamic force within the said artificial joint in its effective range. Yet, the method that implanting the said artificial joint to the target subject is a surgical method, and is not included in the present invention.

The embodiments of the artificial cartilage of the present invention those stated in [0024] those have included supporting fibers those made of piezoelectric material of the said artificial cartilage of the present invention also can be placed into an artificial joint (either during the implanting surgery of the said artificial joint, or, in previous of the said implanting surgery and afterward, together with the said artificial joint, being implanted into the target indication-subject) in order to make the said artificial joint become the joint that can generate adequate Joint-Electricity in daily living alike a healthy natural joint does, and consequently achieve the effect of supplementing the wanted Joint-Electricity to the joint-structure tissues of the said artificial joint and its related muscles. In one embodiment, the said placing and securing of the said artificial cartilage to the said artificial joint is during the implanting surgery that for implanting the said artificial joint into the target subject. However, the method of placing and securing the said artificial cartilage into the said artificial joint during the said implanting surgery is part of the said surgery, and all surgeries are excluded from the present invention. In another embodiment, the said artificial cartilage of the present invention also can be placed into and secured to the artificial joint before the said implanting surgery of the artificial joint, and afterwards, it, together with the said artificial joint, can be implanted into the target indication-subject. The said placing and securing methods before the implanting surgery of the artificial joint include adhesive bonding, and the other methods excluding coating. For example, using the appropriate adhesive bonding material (the type that has included the nanostructured material is one of the examples) that suitable for both the bottom of the said artificial cartilage of the present invention and joint-surface of the said artificial joint to directly bond the said artificial cartilage to the joint-surface of the said artificial joint. After the said artificial joint has been implanted by a proper surgery, it can continuously generate Joint-Electricity alike natural healthy joint does due to it has equipped the said artificial cartilage of the present invention, and which can be subjected to the dynamic force within the said artificial joint in its effective range. Yet, the method that implanting the said artificial joint to the target subject is a surgical method, and is not included in the present invention.

THE ADVANTAGES OF THE ARTIFICIAL CARTILAGE OF THE PRESENT INVENTION over those of the prior art and those without using the present invention, including at least the followings. The artificial cartilage of the present invention has specialty in that it is made of proper piezoelectric material, which is defined in with large enough range of piezoelectric reactivity that can ensure the said artificial cartilage, after the proper implanting surgery for placing it into the target indication-joint (nature joint or artificial joint), can be subjected to the dynamic force within the said joint in its effective range, thus, ensures continuously generating Joint-Electricity. Comparing to that without using the said artificial cartilage, usage of the artificial cartilage of present invention can increase the nurturing on the joint-structure tissues (capsule, ligament, and tendons) of the said joint and its related muscles, thus, achieves the effect of remarkably reducing the pains, increasing muscular strength, and improving the voluntary motor abilities. Besides, through individualized designing the shape of the said artificial cartilage to be appropriate to solving the individualized problems related to defect or lack in cartilage, can prevent it from disturbing the correct bony alignment and joint structure those obtained by the proper surgery for implanting it. This can contribute to ensure it can be subjected to the dynamic force within the said joint in its effective range that obtained by the proper piezoelectric material of the said artificial cartilage of the present invention in order for ensuring the said artificial cartilage can be alike the natural healthy cartilage continuously generating Joint-Electricity during the activities of daily living. Therefore, using the artificial cartilage of the present invention in the indication-joints of the prior art, or, adding the artificial cartilage of the present invention to any artificial joint (which develops to nowadays, still lack of artificial cartilage) can obtain the effect of helping the said joints keeping long term health, promoting their motor functions, preventing the pains that occurred after the similar surgery that using the artificial cartilage of the prior art, remarkably reducing the usage of painkillers, and faster recovering their voluntary motion abilities, prolonging the use life of the said artificial cartilage (and artificial joint), and preventing repeated surgeries. Thus, the artificial cartilage of the present invention is improved and more effective than the artificial cartilage and so-called biomimetic cartilage in the prior art.

While the invention herein disclosed has been described by means of specific embodiments, numerous modifications and variations could be made thereto by those skilled in the art without departing from the scope of the invention set forth in the claims.

What is claimed is:

1. The present invention is an artificial cartilage capable of supplementing Joint-Electricity, the said Joint-Electricity is defined in the intra-articular electricity that generated by the joint-cartilage, and is required by the joint-structure tissues of the said joint and its related muscles for maintaining their normal state, the said artificial cartilage of the present invention has the indication-joints including artificial joints and the natural joints those with lack or defect in cartilage, the human joints those suffering from impairment in generating Joint-Electricity, the said artificial cartilage has a sleek joint-surface and individualized appropriate shape, and is made of the proper piezoelectric material, wherein, the said proper piezoelectric material is defined in the material with the properties of biocompatibility and the piezoelectricity that with a large enough range of piezoelectric reactivity, which is defined in that it can make the said artificial cartilage to have large enough range of piezoelectric reactivity that at least larger than the estimated range of the dynamic force within the target individual indication-joint after the implanting proper surgery, yet, any surgery is not included in the present invention.

2. The artificial cartilage of claim 1, wherein, the said artificial cartilage has individualized appropriate shape, which is obtained through the methods including designing it according to the individualized original healthy cartilage of the said indication-joint, or according to the joint-surface of the target artificial joint, further, for solving the individual problem related to the defect or lack in the cartilage of the indication-joint, the said methods include at least according to the complementary shape of the residual cartilage.

3. The artificial cartilage of claim 1, if necessary, the said artificial cartilage of the present invention can also include supporting fibers, the making method can be, first, making the said piezoelectric material of the said artificial cartilage into the said supporting fibers, which, then, with enough amount, are made into the said artificial cartilage.

4. The artificial cartilage of claim 1, wherein, the sleek joint-surface of the said artificial cartilage can be achieved through the methods including the following, besides the general smoothing processes, it can also be achieved through applying the nanostructurizing process to some of the material that same to that of the said artificial cartilage of the present invention, and then, that after nanostructurizing is coated onto the joint-surface of the said artificial cartilage, or, applying the said nanostructurizing process to enough amount of the material of the said artificial cartilage of the present invention, and then, that after nanostructurizing is made into the said artificial cartilage.

5. The artificial cartilage of claim 3, wherein, the sleek joint-surface of the said artificial cartilage can be achieved through the following methods, which include, at least, first, making the said supporting fibers in the diameter that as small as possible (the smallest diameter that the related industry instantly can make), which, then, with enough amount, are made into the said artificial cartilage of the present invention.

6. The artificial cartilage of claim 1, wherein the said artificial cartilage, not only can be placed and secured into the artificial joint during the implanting surgery for the said artificial joint, but also can be placed and secured into the site corresponding to the joint-surface of the artificial joint before the said implanting surgery through the methods including adhesive bonding the said artificial cartilage of the present invention to the target site of the said artificial joint, coating the material of the said artificial cartilage of the present invention on the said site of the said artificial joint in the shape of the said artificial cartilage of the present invention, and other methods.

7. The artificial cartilage of claim 3, wherein, the said artificial cartilage, not only can be placed and secured into the artificial joint during the implanting surgery for the said artificial joint, but also can be placed and secured into the site corresponding to the joint-surface of the artificial joint before the said implanting surgery through the methods including adhesive bonding the said artificial cartilage of the present invention to the target site of the said artificial joint, and other methods excluding coating.
